SEQUENCE LISTING (1 sequence)

SEQ ID NO: 1 - protein, 1390 aa, Arabidopsis thaliana
  DOMAIN (129)..(272): DEXDc helicase domain
MGSRVPIETIEEDGEFDWEAAVKEIDLACLKTTNASSSSSSHFTPLANPPITANLTKPPAKRQSTLDKFIGRTEHKPENHQVVSECGVNDNDNSPLVGIDPEAAKTWIYPGFVPLRDYQFAITKTALFSNTLVALPTGLGKTLIAAVVMYNYFRWFPQGKIVFAAPSRPLVMQQIEACHNIVGIPQEWTIDLTGQTCPSKRAFLWKSKRVFFVTPQVLEKDIQSGTCLTNYLVCLVIDEAHRALGNYSYCVVVRELMAVPIQLRILALTATPGSKTQAIQGIIDNLQISTLEYRNESDHDVCPYVHDRKLEVIEVPLGQDADDVSKRLFHVIRPYAVRLKNFGVNLNRDIQTLSPHEVLMARDKFRQAPLPGLPHVNHGDVESCFAALITLYHIRKLLSSHGIRPAYEMLEEKLKEGPFARLMSKNEDIRMTKLLMQQRLSHGAPSPKLSKMLEILVDHFKVKDPKTSRVIIFSNFRGSVRDIMNALSNIGDMVKATEFIGQSSGKTLKGQSQKIQQAVLEKFRAGGFNVIVATSIGEEGLDIMEVDLVICFDANVSPLRMIQRMGRTGRKNNGRPLLVLACEGSEKNSYMRKQASGRAIKKHMRNGGTNSFNFHPSPRMIPHVYKPEVQHVEFSIKQFVPRGKKLQEEYATETPAFQKKLTPAETHMLAKYYNNPDEEKLRVSLIAFPHFQTLPSKVHKVMHSRQTGMLIDAMQHLQEPTFSEQSKSFFTEFRAPLGEREELDTGLRVTNDPKDLHSVRDLEVNTSQRKAKQVESPTSTLETTEKDYEESSPTHRYLFSSECASVDTLGNVFVMPVPLLFFPNVLESDNTPLPKTEKQHSCRNTSHIDLVPVDTSEKHRQDNISCKLKERFSPDGASETLETHSLVKRNSTRVGEDDVANSVGEIVLSSDEDDCEGLELSPRLTNFIKSGIVPESPVYDQGEANREEDLEFPQLSSPMRFSNELAGESSFPERKVQHKCNDYNIVSTTTELRTPQKEVGLANGTECLAVSPIPEDWRTPLANLTNTNSSARKDWRVSSGEKLETLRQPRKLKRLRRLGDCSSAVKENYPGITEADHIRSRSRGKKHIRVKWKSAESWFTLSSVAFALVTKKKKMIMDDDVQVFIDEEAEVSSGAEMSADENEDVTGDSFEDSFIDDGTMPTANTQAESGKVDMMAVYRYIQPKISFFYCEVNELIKNHKVSFHRRSLLSQSPLPARFRDLAASSLSPYSAGPLTRINESRSDSDKSLSSLRTPKTTNSESNQDAMMIGNLSVVQISSDSRKRKFSLCNSANAPVINLESKFAAHAQATEKESHEGVRSNAGALEYNDDDDDAFFATLDFDAMEAQATLLLSKQRSEAKEKEDATVIPNPGMQRSDGMEKDAPSFDLGLW

(19) United States
(12) Patent Application Publication
(10) Pub. No.: US 2014/0289902 A1
(43) Pub. Date: Sep. 25, 2014

(54) INCREASING MEIOTIC RECOMBINATION IN PLANTS BY INHIBITING THE FANCM PROTEIN

(71) Applicants: Raphael Mercier, Fontenay Le Fleury (FR); Wayne Crismani, West Des Moines, IA (US)

(72) Inventors: Raphael Mercier, Fontenay Le Fleury (FR); Wayne Crismani, West Des Moines, IA (US)

(21) Appl. No.: 14/343,838

(22) PCT Filed: Sep. 14, 2012
(86) PCT No.: PCT/IB2012/054813

§ 371 Date: Apr. 23, 2014

(30) Foreign Application Priority Data
Sep. 16, 2011 (FR) 1158262

(51) Int. Cl. C12N 15/82

(52) U.S. Cl. CPC C12N 15/8261
USPC 800/285; 435/320.1; 435/419; 800/298

(57) ABSTRACT

The invention relates to a method for increasing the frequency of meiotic recombination in plants by deactivating the FANCM protein, in particular by mutagenesis or silencing of the FANCM gene coding for said protein. The present invention can be used in particular in the field of improving plants and genetic mapping.

The present invention relates to a method for increasing meiotic recombination in plants.

Meiotic recombination is a DNA exchange between homologous chromosomes during meiosis; it occurs during the prophase of the first meiotic division. One of the products of recombination is crossing-over, which leads to an exchange of reciprocal continuity between two homologous chromatids. This prophase (Prophase I) includes 5 successive stages: leptotene, zygotene, pachytene, diplotene, and diakinesis. During leptotene, the chromosomes become individualized, with each chromosome being formed of two sister chromatids originating from the duplication that occurs prior to prophase. During zygotene, the homologous chromosomes pair off, forming a so-called “bivalent” structure that contains four chromatids, two maternal sister chromatids and two paternal sister chromatids, which are homologs of the maternal chromatids. During pachytene, the chromosomes are fully paired-off, and recombination nodules form between the homologous chromatids that are closely linked together by the synaptonemal complex (SC); during diplotene, the SC gradually splits up, and the homologous chromosomes begin to separate, but remain connected at the chiasmata, which correspond to the crossing-over (CO) sites. The chromosomes become condensed during diakinesis; the chiasmata subsist until Metaphase I, during which they maintain the bivalent pairing on either side of the equatorial plate.

Meiotic recombination is triggered by the formation of double-strand breaks (DSB) in either of the homologous chromatids, and results from the repair of these breaks using a chromatid of the homologous chromosome as a matrix.

Meiotic recombination leads to reassortment of paternal- and maternal-origin alleles in the genome, which helps to generate genetic diversity. It therefore is of particular interest in plant improvement programs (WIJNKER & de JONG, Trends in Plant Science, 13, 640-646, 2008). Specifically, improving the recombination rate may make it possible to increase genetic mixing, and therefore the likelihood of producing new combinations of characteristics; it may additionally help to facilitate the introgression of genes of interest, as well as genetic mapping and positional cloning of genes of interest.

Various methods for controlling meiotic recombination have been proposed, based on the overexpression or silencing of any of the many genes identified as being involved or potentially involved in this mechanism. For example, Patent Application WO/0208432 proposes overexpression of the RAD51 protein, which is involved in homologous recombination, in order to stimulate meiotic recombination; U.S. Patent Application 2004023388 proposes overexpressing a meiotic recombination activator selected from: SP011, MRE11, RAD50, XRS2/NBS1, DMC1, RAD51, RPA, MSH4, MSHS, MLH1, RAD52, RAD54, TID1, RAD5S, RADS7, RADS9, a resolvase, a single-stranded DNA binding protein, a protein involved in chromatin remodeling, or a synaptonemal complex protein, in order to increase recombination frequency between homologous chromatids; PCT Patent Application WO 2004016795 proposes increasing recombination between homologous chromosomes by expressing an SPO11 protein merged with a DNA binding domain; PCT Patent Application WO 03104451 proposes increasing the recombination potential among homologous chromosomes by the overexpression of a protein (MutS) involved in mismatch repair.

However, in most cases, the effect of these candidate genes on the formation of meiotic COs in planta has not been confirmed, and other genes that are active in this phenomenon must therefore be identified.

One of the factors known to affect the meiotic recombination rate is the interference phenomenon: The formation of a CO at one location of the chromosome inhibits the formation of other COs nearby. However, it has recently been shown that 2 distinct meiotic CO formation pathways in fact exist (HOLLINGSWORTH & BRILL, Genes Dev., 18, 117-125, 2004; BERCHOWITZ & COPENHAVER, Current Genomics, 11, 91-102, 2010). The first one generates interfering COs referred to as Type I COs (COI), and involves a set of genes collectively designated under the gene names ZMM (ZIP1, ZIP2, ZIP3, ZIP4, MER3, MSH4, MSH5) along with the MLH1 and MLH3 proteins; the second pathway generates noninterfering COs, referred to as Type II COs (COII), and is dependent upon the MUS81 gene.

The use of either of these two pathways is variable from one organism to another. In higher plants, represented by the model plant Arabidopsis thaliana, the two pathways coexist, with the Type I CO pathway being predominant; it has been observed that inactivation of the AtMSH4, AtMSH5, atMER3, SHOC1, or AtZIP4 genes (respective homologs in Arabidopsis thaliana of the MSH4, MSH5, MER3, ZIP2, and ZIP4 genes) induced up to an 85% reduction in CO frequency (HIGGINS et al., 2004, previously cited; MERCIER et al., 2005, previously cited; CHELYSHEVA et al., PloS Genet. 3, e83, 2007; HIGGINS et al., Plant J., 55, 28-39, 2008; MACAISNE et al., Current Biology, 18, 1432-1437, 2008); this decrease is accompanied by a considerable reduction in fertility.

The Inventors have now discovered that inactivating the “Fanconi Anemia Complementation Group M” (FANCM) gene of Arabidopsis thaliana (AtFANCM) gene compensates for the effects of inactivating AtMSH5, SHOC1, or AtZIP4, and makes it possible to increase the number of meiotic COs and to restore fertility in zmm Atzip4−/−, Atmsh5−/−, and Atshoc1−/− mutants. They have also discovered that the effects of inactivating the FANCM gene on increasing the number of meiotic COs occurred not only in zmm mutants, but also in wild-type plants having functional ZMM genes.

The FANCM protein was initially identified in humans in the context of searching for mutations associated with Fanconi anemia, which is a genetic disease characterized by genomic instability and a predisposition to cancer. This protein helps to repair DNA lesions; the human FANCM protein contains two helicase domains in its N-terminal half: a DEXDc domain (cd00046), and a HELICc domain (cd00079) as well as an ERCC1/XPF endonuclease domain in its C-terminal half; however, the latter domain is probably not functional because it is degenerate for several residues essential to endonuclease activity (MEETEI et al., Nat. Genet., 37, 958-963, 2005).

The FANCM protein appears to be preserved, and various homologs of this protein have been identified in eukaryotes based on sequence homologies. Vertebrate FANCM proteins, like human FANCM, have the helicase domains and the endonuclease domain; drosophila FANCM, as well as the FANCM homolog in Saccharomyces cerevisiae yeast (referred to as MPH1 for “Mutator Phenotype 1”), are shorter and only have the helicase domains. Plant FANCMs, represented by the Arabidopsis thaliana protein, likewise do not have the endonuclease domain. AtFANCM is coded by the AT1G35530 gene; two predicted isoforms of this protein are described in the sequence databases: one (GenBank: NP_{—}001185141; UniProtKB: F4HYE4), represented in the attached sequence list under the number SEQ ID NO: 1, has a size of 1390 amino acids; the other (GenBank: NP_{—}174785; UniProtKB: F4HYE5), has a size of 1324 amino acids. These two isoforms differ from one another by the presence of two insertions in the NP_{—}001185141/F4HYE4 sequence: one of 45 amino acids between the 575 and 576 positions of the NP_{—}174785/F4HYE5 sequence, and the other of 21 amino acids between the 1045 and 1046 positions of the NP_{—}174785/F4HYE5 sequence.

Regardless of the isoform involved, AtFANCM has, in its N-terminal half, the two helicase domains DEXDc (amino acids 129-272 of SEQ ID NO: 1) and HELICc (amino acids 445-570 of SEQ ID NO: 1); these two domains are respectively described under the reference numbers cd00046 and cd00079 in the CDD database (MARCHLER-BAUER et al., Nucleic Acids Res. 39 (D) 225-9, 2011).

A search in the sequence databases identified AtFANCM orthologs in a large panel of eukaryotes, and it can be assumed that this protein is preserved in all higher plants.

By way of non-limiting examples of AtFANCM orthologs, we list the following;

the Vitis vinifera protein whose polypeptide sequence is available in the GenBank database under access number CBI18266;

the Physcomitrella patens protein whose polypeptide sequence is available in the Phytozome database under access number Ppls9_{—}477V6;

the Ricinus communis protein whose polypeptide sequence is available in the GenBank database under access number XP_{—}002526811;

the Oryza sativa protein whose polypeptide sequence is available in the GenBank database under access number AAX96303;

the Populus trichocarpa protein whose polypeptide sequence is available in the Phytozome database under access number POPTR_{—}0013s11390.

The goal of the present invention is a method for increasing the frequency of meiotic COs in a plant, wherein it includes the inhibition in said plant of a protein referred to hereinafter as FANCM, with said plant having at least 30%, and by order of increasing preference at least 35, 40, 45, 50, 55, 60, 65, 70, 75, 80, 85, 90, 95, or 98% sequence identity, or at least 45%, and by order of increasing preference, at least 50, 55, 60, 65, 70, 75, 80, 85, 90, 95, or 98% of sequence similarity with the AtFANCM protein of sequence SEQ ID NO: 1, and containing a DEXDc (cd00046) helicase domain and a HELICc (cd0079) helicase domain.

According to a preferred embodiment of the present invention, the DEXDc helicase domain of said FANCM protein has at least 65%, and by order of increasing preference, at least 70, 75, 80, 85, 90, 95 or 98% sequence identity, or at least 75%, and by order of increasing preference, at least 80, 85, 90, 95, or 98% sequence similarity with the DEXDc domain of the AtFANCM protein (amino acids 129-272 of SEQ ID NO: 1).

According to another preferred embodiment of the present invention, the HELICc helicase domain of said FANCM protein has at least 60%, and by order of increasing preference, at least 65, 70, 75, 80, 85, 90, 95, or 98% sequence identity, or at least 70%, and by order of increasing preference, at least 75, 80, 85, 90, 95, or 98% sequence similarity with the HELICc domain of the AtFANCM protein (amino acids 445-570 of SEQ ID NO: 1).

The sequence identity and similarity values listed here are calculated by using the BLASTP program or the Needle program with the default parameters. Similarity calculations are performed by using the BLOSUM62 matrix.

Inhibition of the FANCM protein can be obtained by suppressing or decreasing its activity or by suppressing or decreasing the expression of the corresponding gene.

Specifically, inhibition can be obtained via mutagenesis of the FANCM gene. For example, a mutation in the coding sequence can induce, depending upon the nature of the mutation, expression of an inactive protein, or of a reduced-activity protein; a mutation at a splicing site can also alter or abolish the protein's function; a mutation in the promoter sequence can induce the absence of expression of said protein, or the decrease of its expression.

Mutagenesis can be performed, e.g., by suppressing all or part of the coding sequence or of the FANCM promoter, or by inserting an exogenous sequence, e.g., a transposon or a T-DNA, into said coding sequence or said promoter. It can also be performed by inducing point mutations, e.g., using EMS mutagenesis or radiation.

The mutated alleles can be detected, e.g., by PCR, by using specific primers of the FANCM gene.

Various high-throughput mutagenesis and splicing methods are described in the prior art. By way of examples, we note “TILLING” (Targeting Induced Local Lesions In Genome)-type methods, described by McCALLUM et al., Plant Physiol., 123, 439-442, 2000). The absence of FANCM functionality in mutants can be verified based on the phenotypic characteristics of their offspring; homozygous plants for a mutation inactivating the FANCM gene have a meiotic CO rate that is higher than that of the wild plants (not carrying the mutation in the FANCM gene) from which they originated. Generally, this meiotic CO rate is at least 2 times higher, preferably at least 3 times higher than that of the wild plants from which they originated.

Mutant plants containing a mutation in the FANCM gene that induces inhibition of the FANCM protein are also part of the goal of the present invention, except for plants of the Arabidopsis thaliana species, in which said mutation is an insertion of T-DNA into said gene. This mutation can be, e.g., a deletion of all or part of the coding sequence or of the FANCM promoter, or it may be a point mutation of this coding sequence or of this promoter.

Advantageously, inhibition of the FANCM protein is obtained by silencing the FANCM gene. Various techniques for silencing genes in plants are known (for review, see, e.g.: WATSON & GRIERSON, Transgenic Plants: Fundamentals and Applications (Hiatt, A., ed.) New York: Marcel Dekker, 255-281, 1992; CHICAS & MACINO, EMBO Reports, 21, 992-996, 2001). Antisense inhibition or co suppression, described, e.g., in U.S. Pat. No. 5,190,065 and U.S. Pat. No. 5,283,323, is noteworthy. It is also possible to use ribozymes targeting the mRNA of the FANCM protein.

Preferably, silencing of the FANCM gene is induced by RNA interference targeting said gene.

An interfering RNA (iRNA) is a small RNA that can silence a target gene in a sequence-specific way. Interfering RNA include, specifically, “small interfering RNA” (siRNA) and micro-RNA (miRNA).

Initially, DNA constructions for expressing interfering RNA in plants contained a fragment of 300 pb or more (generally from 300 to 800 pb) of the cDNA of the target gene, under the transcriptional control of an appropriate promoter. At present, the most widely-used constructions are those that can produce a hairpin RNA transcript. In these constructions, the fragment of the target gene is inversely repeated, with generally a spacing region between the repetitions (for review, cf. WATSON et al., FEBS Letters, 579, 5982-5987, 2005). It is also possible to use artificial micro-RNA (amiRNAs) directed against the FANCM gene (OSSOWSKI et al., The Plant Journal, 53, 674-690, 2008; SCHWAB et al., Methods Mol Biol., 592, 71-88, 2010; WEI et al., Funct Integr Genomics., 9, 499-511, 2009).

The goal of the present invention is recombinant DNA constructions, specifically expression cassettes, producing an iRNA that silences the FANCM gene.

An expression cassette of the invention includes a recombinant DNA sequence whose transcript is an iRNA, specifically an hpRNA or an amiRNA, targeting the FANCM gene, placed under transcriptional control of a functional promoter in a plant cell.

A broad selection of appropriate promoters for expressing heterologous genes in plant cells or in plants is available in the prior art.

These promoters can be obtained, e.g., from plants, plant viruses, or bacteria such as Agrobacterium. They include constitutive promoters namely, promoters that are active in most tissues and cells and under most environmental conditions as well as tissue-specific or cell-specific promoters, which are only active or primarily active in certain tissues or certain types of cells, and inducible promoters that are activated by physical or chemical stimuli.

Examples of constitutive promoters that are currently used in plant cells are the 35S promoter of the cauliflower mosaic virus (CaMV) described by KAY et al. (Science, 236, 4805, 1987), or derivatives thereof, the cassava vein mosaic virus (CsVMV) described in International Patent Application WO 97/48819, the maize ubiquitin promoter, or the rice “Actin-Intron-actin” promoter (McELROY et al., Mol. Gen. Genet., 231, 150-160, 1991; GenBank access number S 44221).

In the scope of the present invention, a meiosis-specific promoter (that is, one that is active exclusively or preferably in cells undergoing meiosis) can also be used. By way of non-limiting example, the DMC1 promoter (KLIMYUK & JONES, Plant J., 11, 1-14, 1997) is noteworthy.

The expression cassettes of the invention generally include a transcriptional terminator, e.g., the 3′NOS nopaline synthase terminator (DEPICKER et al., J. Mol. Appl. Genet., 1, 561-573, 1982), or the 3′CaMV terminator (FRANCK et al., Cell, 21, 285-294, 1980). They may also include other transcription-regulating elements such as amplifiers.

The recombinant DNA constructions of the invention also encompass recombinant vectors containing an expression cassette of the invention. These recombinant vectors may also include one or several marker genes, which enable the selection of the transformed cells or plants.

The selection of the most appropriate vector depends, in particular, on the expected host and on the anticipated method to be used for transforming the relevant host. Numerous methods for genetic transformation of plant cells or plants are available in the prior art, for numerous dicotyledonous or monocotyledonous plant species. By way of non-limiting examples, we may mention virus-mediated transformation, transformation by microinjection or by electroporation, transformation by microprojectiles, transformation by Agrobacterium, etc.

Another goal of the invention is a host cell including a recombinant DNA construction of the invention. Said host cell can be a prokaryote cell, e.g., an Agrobacterium cell, or a eukaryote cell, e.g., a plant cell that has been genetically transformed by a DNA construction of the invention. The construction can be expressed transiently, it can also be incorporated into a stable extrachromosomal replicon, or integrated into the chromosome.

The invention additionally provides a method for producing a transgenic plant that has a meiotic CO higher than that of the wild plant from which it originated, wherein it includes the following steps:

transforming a plant cell with a vector containing an expression cassette of the invention;

culturing said transformed cell in order to regenerate a plant that has in its genome a transgene containing said expression cassette.

The invention also encompasses plants that have been genetically transformed by a DNA construction of the invention. Preferably, said plants are transgenic plants, inside which said construction is contained in a transgene integrated into the plant's genome, such that it is transmitted to following plant generations. The expression of the DNA construction of the invention results in a negative regulation of the FANCM gene's expression, which confers to said transgenic plants a meiotic CO rate that is higher than that of the wild plants (not containing the DNA construction of the invention) from which they originated. Generally, this meiotic CO rate is at least 2 times higher, preferably at least 3 times higher than that of the wild plants from which they originated.

The present invention can be applied in the field of plant improvement in order to accelerate the production of new varieties. It also facilitates crossbreeding between related species, and therefore the introgression of useful traits. It also helps to accelerate the establishment of genetic maps and positional clonings.

The present invention applies to a broad range of monocotyledonous or dicotyledonous agronomically-interesting plants. By way of non-limiting examples, we may mention canola, sunflower, potatoes, maize, wheat, barley, rye, sorghum, rice, beans, carrots, tomatoes, zucchini, bell peppers, eggplants, turnips, onions, peas, cucumbers, leeks, artichokes, beets, cabbage, cauliflower, salad greens, endive, melons, watermelons, strawberry plants, apple trees, pear trees, plum trees, poplars, grapevines, cotton, roses, tulips, etc.

The present invention will be more fully understood through the following descriptive supplement, which refers to non-limiting examples illustrating the effects of mutations of the AtFANCM gene on meiotic recombination and CO rate.

### EXAMPLE 1

### Production of Mutants That Suppress Zmm Gene Mutations

Homozygous Arabidopsis thaliana seeds for inserting T-DNA into ZIP4, SHOC1, or MSH5 (Atzip4−/−, Atmsh5−/−, or Atshoc1−/−) were mutated by EMS (ethyl methane sulfonate). The plants that grew from the mutated seeds (population Ml, heterozygous for mutations induced by EMS, and homozygous for mutation of the ZMM gene) have an identical phenotype, resulting from inactivation of the ZMM gene, which results in a marked decrease in CO frequency and a sharp drop in fertility (“semisterile” plants), resulting in the formation of short siliquae that are easily differentiated from those of wild plants. The M1 plants were autopollinated in order to produce a population of offspring (population M2) potentially containing homozygous plants for the EMS-induced mutations. Plants from population M2 that had a longer siliqua than that of homozygous plants from population M1 were selected and genotyped in order to verify their homozygous status for the insertion of T-DNA into the relevant ZMM gene. The segregation of their chromosomes during meiosis was compared to that of wild plants and non-EMS-mutated homozygous zmm plants.

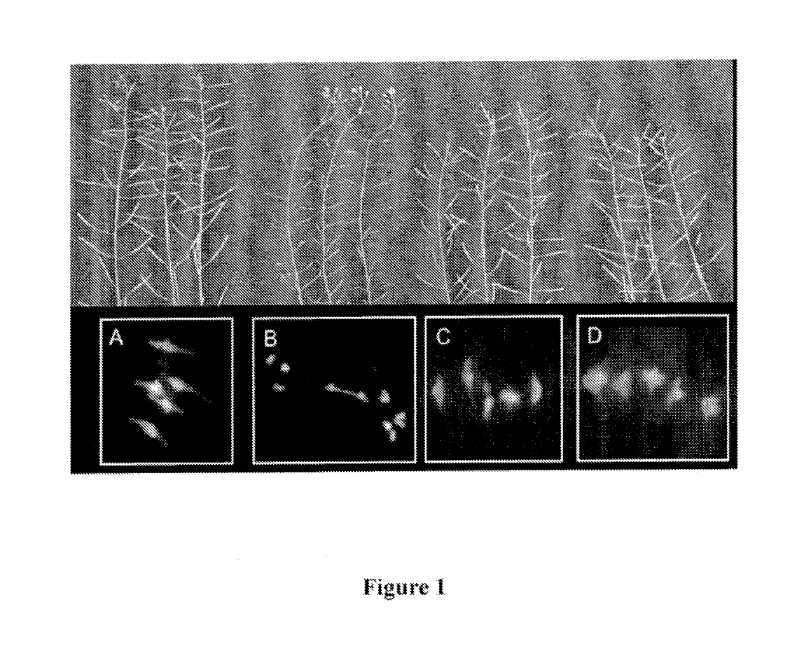

The results are illustrated in FIG. 1.

Legend for FIG. 1: The top of the figure shows the length of the siliquae of the various compared plants. The boxes at the bottom of the figure illustrate chromosome segregation during Metaphase I. A: ZMM/SUPPRESSOR: wild plant; B: zmm/SUPPRESSOR: homozygous plant for zmm mutation and wild for EMS mutation; C: zmm/suppressor: homozygous plant for zmm mutation and for EMS-induced mutation; D: ZMM/suppressor: plant not carrying the zmm mutation and homozygous for the EMS-induced mutation.

In the zmm/SUPPRESSOR plants, we observe poor chromosome segregation that results from a decrease in CO formation and therefore of bivalents, induced by the zmm mutation, and that leads to nonviable, unbalanced gametes. Conversely, in the zmm/suppressor and ZMM/suppressor plants as in the wild plants, we see normal chromosome segregation, identical to that of the wild plants, and resulting in the formation of balanced gametes.

The fertility of the ZMM/suppressor plants is identical to that of the wild plants. Moreover, they do not present any visible phenotypic differences from the wild plants. The EMS-induced mutations are recessive. Indeed, they do not take the form of a detectable phenotype in the heterozygous state in the M1 mutant population, and the plants resulting from backcrossing of the zmm/suppressor mutants with the initial zmm mutants from which they respectively originated have a phenotype that does not differ from that of the initial mutants. Additionally, segregation of the “fertile” and “semisterile” phenotypes in offspring originating from autopollination of plants resulting from backcrossing of the zmm/suppressor mutants with the initial zmm mutants from which they respectively originated indicates that a single locus is involved in the mutation.

### EXAMPLE 2

### Localization of Mutations in the FANCM Gene

Among the EMS mutants described in Example 1 above, five lines of ZIP4-gene mutation-suppressor mutants, 3 lines of SHOC1-gene mutation-suppressor mutants, and one line of MSH5-gene mutation-suppressor mutants were isolated. These lines are named, respectively: zip4(s)1), zip4(2)3, zip4(s)6, zip4(2)7, zip4(s)8, shoc1(s)8, shoc1(s)14, shoc1(s)38, and msh5(s)59.

The corresponding mutations were all localized in a single gene (At1g35530), a homolog of the FANCM (Fanconi Anemia Complementation Group M) gene. Complementation tests confirmed that these mutations were in fact responsible for the observed phenotype.

Table I below lists the nature and position of the identified mutations.

TABLE I

| | | Effect of mutation/position |
|---|---|---|
| Allele Names | | in relation to FANCM protein |
| zip4(s)1 | fancm-1 | G510D substitution |
| zip4(s)3 | fancm-2 | Mutation of exon 17 splicing acceptor site/I621 |
| zip4(s)6 | fancm-3 | Mutation of exon 13 splicing acceptor site/R481 |
| zip4(s)7 | fancm-4 | E586K substitution |
| zip4(s)8 | fancm-5 | G138R substitution |
| shoc1(s)8 | fancm-6 | S535F substitution |
| shoc1(s)14 | fancm-7 | Mutation of exon 2 splicing acceptor site/D17 |
| msh5(s)59 | fancm-8 | G402E substitution |

### EXAMPLE 3

### Influence of Inactivating the FANCM Gene on Meiotic Recombination Frequency

The meiotic recombination frequency in FANCM-ZIP4 (wild plants), FANCM/zip4 (homozygous for the zip4 mutation and non-mutated in FANCM), fancm-1/ZIP4 (homozygous for the fancm-1 mutation and non-mutated in ZIP4), and fancm-1/zip4 (homozygous for the 2 zip4 and fancm-1 mutations) plants was compared.

The meiotic recombination frequency was measured by tetrad analysis using fluorescent markers, as described by BERCHOWITZ & COPENHAVER (Nat. Protoc., 3, 41-50, 2008).)

The genetic distance in 2 adjacent intervals on Chromosome 5 (I5c and I5d) was measured in FANCM/ZIP4, FANCM/zip4, fancm-1/zip4, or fancm-1/ZIP4 plants.

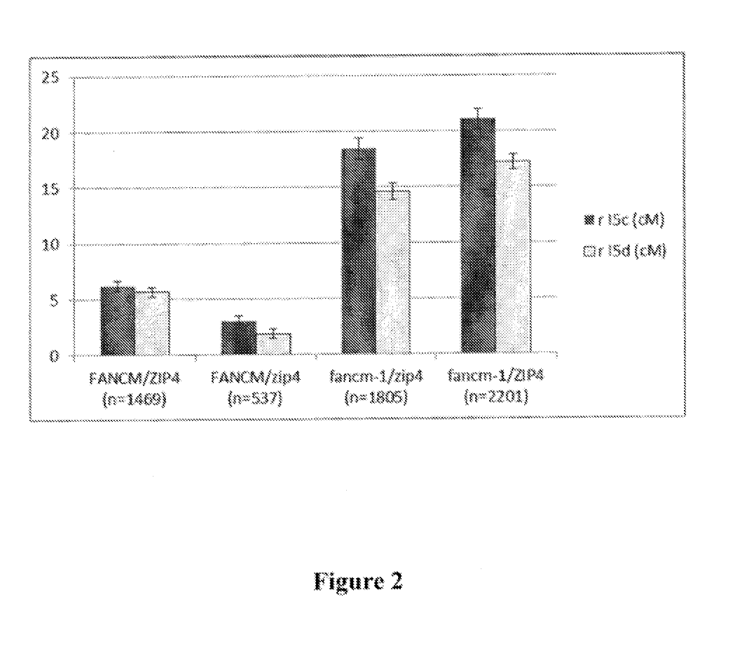

The results are illustrated in FIG. 2.

Legend for FIG. 2:

X-axis: genotype of tested plants; the number of tetrads tested for each genotype is indicated in parentheses.

Y-axis: genetic distance (in cM).

In black: genetic distance measured for the I5c interval; in light grey: genetic distance measured for the I5d interval.

I5c and I5d measure, respectively, 6.19 cM and 5.65 cM in the FANCM/ZIP4 plants and only 2.98 cM and 1.86 cM in the FANCM/zip4 plants. However, the genetic distance is greatly increased in the fancm/zip4 plants (18.45 cM and 14.57 cM for I5c and I5d, respectively) and even more so in the case of fancm/ZIP4 (21.06 cM and 17.20 cM for I5c and I5d, respectively).

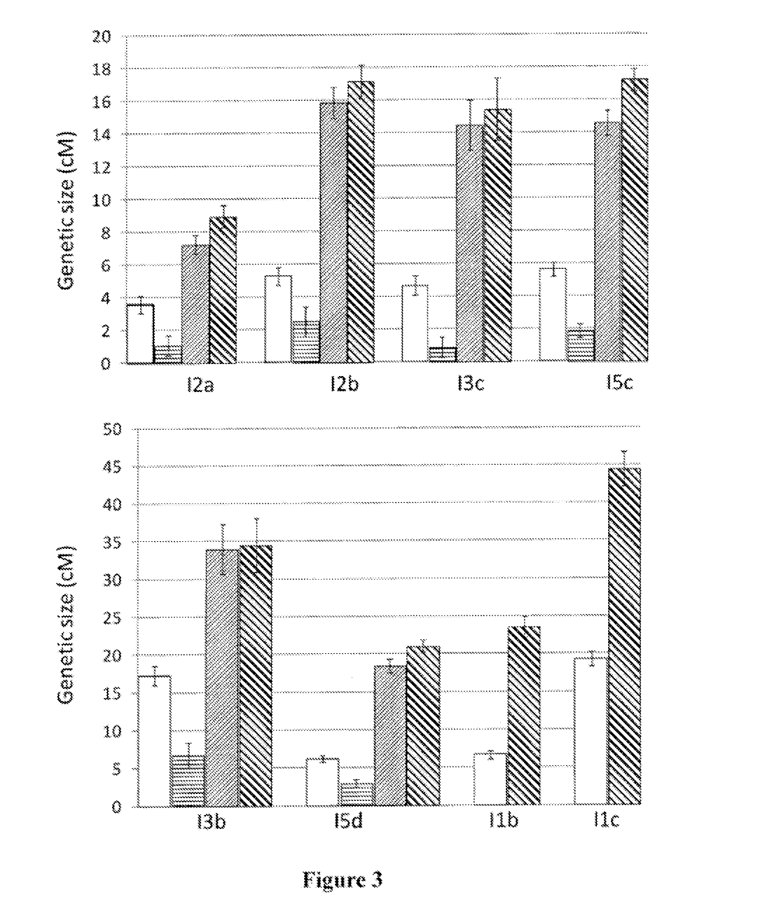

In another series of experiments, the genetic distance was measured over a larger number of adjacent intervals, located on Chromosome 1 (I1b and I1c), Chromosome 2 (I2a and I2b), Chromosome 3 (I3b and I3c), and Chromosome 5 (I5c and I5d). The results are illustrated in FIG. 3.

Legend for FIG. 3:

□: FANCM/ZIP4

[symbol] : FANCM/zip4

[symbol] : fancm-1/zip4

[symbol] : fancm-1/ZIP4

X-axis: tested interval.

Y-axis: genetic distance (in cM).

Recombination is reduced by a factor of 2 to 3 in the FANCM/zip4 mutant compared to the wild type. In fancm-1/zip4, the distances are increased by a factor of 1.9 to 3.1 compared to the wild type (P<10⁻⁵). This confirms that the FANCM mutation not only restores CO formation in the absence of ZIP4, but also increases CO frequency well beyond that observed in the wild plants. In fancm-1/ZIP4, recombination is increased even more than in fancm-1/zip4, on average by 12% (P<0.05 in 3 individual intervals out of 6). Hence, if we compare the fancm-1 mutant to the wild type, the genetic distance is increased by a factor of 2 to 3.6 (P<10⁻⁸) over the 8 tested intervals, which emphasizes the importance of FANCM in limiting CO frequency.

1. A method for increasing meiotic CO frequency in a plant, including the inhibition is said plant of a Fanconi Anemia Complementation Group M (FANCM) protein, wherein said protein contains a DEXDc helicase domain (cd00046) and a HELICc helicase domain (cd00079), and has at least 30% sequence identity with the amino acid of SEQ ID NO: 1.

2. The method according to claim 1, wherein the DEXDc helicase domain of said FANCM protein has at least 65% sequence identity with the DEXDc domain of amino acids 129-272 of SEQ ID NO: 1.

3. The method according to claim 1, wherein the HELICc helicase domain of said FANCM protein has at least 60% sequence identity with the HELICc domain of amino acids 445-570 of SEQ ID NO: 1.

4. The method according to claim 1, wherein the inhibition of the FANCM protein is obtained by mutagenesis of the FANCM gene or of its promoter.

5. The method according to claim 1, wherein the inhibition of the FANCM protein is obtained by silencing the FANCM gene by expressing an interfering RNA inside said plant that targets said gene.

6. An expression cassette comprising a recombinant DNA sequence whose transcript is an interfering RNA targeting the FANCM gene placed under transcriptional control of a functional promoter in a plant cell.

7. The expression cassette according to claim 6, wherein said interfering RNA is selected from:
an hpRNA targeting the FANCM gene; or
an amiRNA targeting the FANCM gene.

8. A recombinant vector containing an expression cassette according to claim 6.

9. A host cell containing an expression cassette according to claim 6.

10. A mutant plant containing a mutation in the FANCM gene, with said mutation inducing the inhibition of the FANCM protein inside said plant except for a mutant plant of the Arabidopsis thaliana species, in which said mutation is a T-DNA insertion inside said gene.

11. A transgenic plant containing a transgene including an expression cassette according to claim 6.

12. A recombinant vector containing an expression cassette according to claim 6, wherein said interfering RNA is selected from:
an hpRNA targeting the FANCM gene; or
an amiRNA targeting the FANCM gene.

13. A host cell containing an expression cassette according to claim 6, wherein said interfering RNA is selected from:
an hpRNA targeting the FANCM gene; or
an amiRNA targeting the FANCM gene.

14. A transgenic plant containing a transgene including an expression cassette according to claim 6, wherein said interfering RNA is selected from:
an hpRNA targeting the FANCM gene; or
an amiRNA targeting the FANCM gene.

15. The method of claim 1 wherein the FANCM protein has at least 90% sequence identity with the amino acid sequence SEQ ID NO: 1.

16. The method of claim 1 wherein the FANCM protein has at least 95% sequence identity with the amino acid sequence SEQ ID NO: 1.

17. The method of claim 1 wherein the FANCM protein has the amino acid sequence SEQ ID NO:1.
